(19) United States
(12) Patent Application Publication
(10) Pub. No.: US 2026/0234524 A1
(43) Pub. Date: Aug. 13, 2026

(54) A METHOD OF TRANSDUCTION

(71) Applicant: Oribiotech LTD, London, Greater London (GB)

(72) Inventors: Farlan Veraitch, London (GB); William Raimes, Philadelphia, PA (US); Arman Amini, London (GB); Shaun Mansfield, London (GB); Aoife Hayes, Cork (IE); Hamza Patel, Hitchin (GB); Ana-Maria lonescu, London (GB)

(21) Appl. No.: 19/150,784

(22) PCT Filed: Jan. 25, 2024
(86) PCT No.: PCT/GB2024/050204

§ 371 Date: Jul. 24, 2025

(30) Foreign Application Priority Data
Jan. 26, 2023 (GB) 2301148.9

(51) Int. Cl. C12M 1/00; C12M 1/02; C12M 1/04; C12M 1/12; C12M 3/04; C12M 3/06; C12N 5/0783; C12N 15/86

(52) U.S. Cl. CPC C12M 23/26; C12M 23/06; C12M 23/24; C12M 27/00; C12M 27/10; C12M 27/16; C12N 5/0636; C12N 15/86; C12N 2527/00; C12N 2740/15043

(57) ABSTRACT

A method of transducing T cells, comprising providing a compressible container including a base, a top arranged substantially in parallel to the base, and at least one flexible wall element extending between the top and the base and defining an internal volume of the compressible container. A population of T cells in a cell processing medium is added into the internal volume. A transduction agent is added into the internal volume. The base of the compressible container is moved with respect to the top, thereby causing turbulence of the contents in the internal volume, for a first period of time.

### CROSS-REFERENCE TO RELATED APPLICATIONS

This application is a national phase entry under 35 U.S.C. § 371 of International Patent Application PCT/GB 2024/050204, filed Jan. 25, 2024, designating the United States of America and published as International Patent Publication WO 2024/157020 A1 on Aug. 2, 2024, which claims the benefit under Article 8 of the Patent Cooperation Treaty of Great Britain Patent Application Serial No. 2301148.9, filed Jan. 26, 2023.

### TECHNICAL FIELD

The disclosure relates to a method of transducing T cells within a compressible container. More particularly, the disclosure relations to a method of transducing T cells, such as CD3+ T cells with CD4+ markers and/or CD3+ T cells with CD8+ markers, within a compressible container. The method of transduction discussed herein may form part of a cell and/or gene therapy manufacturing process.

### BACKGROUND

Cell and gene therapy manufacturing processes are often complex and include manual or semi-automated steps across several devices. Cells are “living” entities sensitive to even the simplest manipulations (such as differences in a cell transferring procedure). The role of cell manufacturing equipment in ensuring scalability and reproducibility is an important factor for cell and gene therapy manufacturing.

Cell-based therapeutic products (CTP) have gained significant momentum in recent years, and thus there is a need for improved cell manufacturing equipment and methods for various cell manufacturing procedures, for example, generation of chimeric antigen receptor (CAR) T cells, and various cell manufacturing processes such as collection, purification, transduction, incubation/recovery, washing, infusion into patient and/or freezing.

Transduction of cells is a process of introducing genetic material into a cell through the use of a transduction agent. This process confers specificity for target antigens to the T cells. Transduction may also confer other favorable characteristics to the T cells, such as improving proliferation, cytokine production, activation signal and effector function characteristics of the T cells. CAR T cells are typically used for cell therapy in the treatment of cancer. CAR T cells are generated by transducing T cells, thereby genetically altering the T cells to produce CARs that bind to cancerous cells.

Typical methods of transducing T cells involve first isolating a patient's T cells. The T cells and a transduction agent are then added to a culture container, such as a flask or a bag. As the transduction agent interacts with the T cells, genetic material is transferred to the T cells to transduce the T cells. This process may be encouraged by mixing the T cell and transduction agent mixture, such as by stirring the mixture manually or via an impeller. Alternatively, the T cell and transduction agent mixture can be provided in a rigid culture container and may be mixed manually or by placing the culture container on a shaker plate. Generally, the transduction of cells is commonly undertaken within a minimal amount of processing medium, to encourage interactions between the transduction agent and the cells contained therein. The transduced CAR T cells are then infused into the patient where they will target the antigens presenting in cancer cells.

The culture or further processing of transduced cells typically requires the addition of further medium and nutrients to the processing medium in order to maintain a desired level of cell growth. However, devices utilized for transduction, including shaker flasks, roller bottles, T-flasks and bags, have a limited, and usually minimal, working volume. Accordingly, the transduced cells are typically required to be transferred to a container having a larger working volume.

A key limiting factor in the production of cells or gene therapies for use in medicine is the absence of compact, automated closed systems for performing unit operations without contamination. The operating systems are largely manual and hence expensive to operate. Multiple pieces of equipment, particularly containers, are typically required to cover all of the non-cell culture steps, which involves many transfers, each of which is an opportunity for operator errors and contamination to occur. Furthermore, with increasing manual operations comes increasing risk of manual errors and therefore the current labor-intensive processes lack the robustness required for the manufacture of clinical-grade therapeutics.

There is therefore a need for improved methods of transduction that provide a greater transduction efficiency over the conventional methods of transduction. In other words, there is a need for a greater number of cells to be transduced in comparison with conventional methods of transduction. In particular, it is noted that current methods of transducing T cells generally have low transduction efficiencies. Typically, only a subset of target cells is transduced with the desired genetic material. For example, only 5% to 35% of the cells are typically successfully transduced.

There is also a need for methods of transduction, and associated apparatuses and devices, in which the transfer of cells between various containers and/or equipment is mitigated or negated entirely.

There is also a need for the automation, or semi-automation, of transduction methods and further cell processing steps.

### BRIEF SUMMARY

According to a first aspect of the disclosure, there is provided a method of transducing T cells, comprising:
providing a compressible container including a base, a top arranged substantially in parallel to the base, and at least one flexible wall element extending between the top and the base and defining an internal volume of the compressible container;
adding a population of T cells in a cell processing medium into the internal volume;
adding a transduction agent into the internal volume; and
moving the base with respect to the top, thereby causing turbulence of contents in the internal volume, for a period of time, such as a first period of time.

The movement of the base of the compressible container causes turbulence of the contents in the internal volume of the compressible container. This turbulence suspends and disperses the T cells in the cell processing medium to increase the instances of contact between the transduction agent and the T cells in the solution, thereby increasing transduction efficiency. Additionally, the provision of a compressible container enables a variable working volume within a single container, thus mitigating the need for additional containers and the associated transfer to such containers. Moreover, a compressible container enables fluid manipulations and hence facilitates further processing steps.

According to a second aspect of the disclosure, there is provided a method of transducing T cells, comprising the ordered steps of:
i) providing a compressible container including a base, a top arranged substantially in parallel to the base, and at least one flexible wall element extending between the top and the base and defining an internal volume of the compressible container;
ii) adding a population of T cells in a cell processing medium into the internal volume;
iii) continuously moving the base with respect to the top, thereby causing turbulence of contents in the internal volume, at a first rate for a first period of time;
iv) optionally adding further cell processing medium into the internal volume;
v) continuously moving the base with respect to the top, thereby causing turbulence of contents in the internal volume, at a second rate for a second period of time, the second rate being greater than the first rate;
vi) optionally adding further cell processing medium into the internal volume;
vii) continuously moving the base with respect to the top, thereby causing turbulence of contents in the internal volume, for a third period of time, wherein the method further comprises the step of adding a transduction agent into the internal volume during step ii) or step iii).

Advantageously, this provides for continuous mixing of the contents of the compressible container. Continuous mixing of the compressible container allows the T cells to continuously remain suspended in cell processing medium, thereby allowing for effective growth of the T cells, and also enables dispersion of the T cells with the transduction agent during periods of mixing to increase interaction between the T cells and the transduction agent for effective transduction of the T cells. In this way, the desired cellular growth and transduction efficiency can be achieved.

In examples, the method is further defined where:
step iii) comprises continuously rotating the base about an axis of rotation extending within a horizontal plane defined by the base at the first rate;
step v) comprises continuously rotating the base about an axis of rotation extending within a horizontal plane defined by the base at the second rate; and
step vi) comprises continuously translating the base toward the top along a central longitudinal axis of the compressible container, thereby compressing the compressible container.

In examples, the method is provided as a method of transducing and culturing T cells.

According to a third aspect of the disclosure, there is provided a method of transducing T cells, comprising the ordered steps of:
i) providing a compressible container including a base, a top arranged substantially in parallel to the base, and at least one flexible wall element extending between the top and the base and defining an internal volume of the compressible container;
ii) adding a population of T cells in a cell processing medium into the internal volume;
iii) optionally maintaining the base stationary with respect to the top following the addition of the transduction agent for a predetermined period of time;
iv) moving the base with respect to the top, thereby causing turbulence of contents in the internal volume, for a first period of time;
v) maintaining the base stationary with respect to the top for a second period of time;
vi) repeating steps iv) and v) for a predetermined period of time; and
vii) adding a transduction agent into the internal volume during any of steps ii) to vi).

Advantageously, this provides for intermittent mixing of the contents of the compressible container. Intermittent mixing of the compressible container allows the T cells to settle on the base during periods of rest, thereby allowing for effective growth of the T cells, and also enables resuspension and dispersion of the T cells with the transduction agent during periods of mixing to increase interaction between the T cells and the transduction agent for effective transduction of the T cells. In this way, the appropriate balance between cellular growth and transduction efficiency can be achieved.

According to a fourth aspect of the disclosure, there is provided a method of transducing and culturing T cells, comprising the ordered steps of:
i) providing a compressible container including a base, a top arranged substantially in parallel to the base, and at least one flexible wall element extending between the top and the base and defining an internal volume of the compressible container;
ii) adding a population of T cells in a cell processing medium into the internal volume;
iii) optionally maintaining the base stationary with respect to the top following the addition of the transduction agent for a predetermined period of time;
iv) intermittently moving the base with respect to the top, thereby intermittently causing turbulence of contents in the internal volume, for a first period of time;
v) adding a transduction agent into the internal volume during the step of adding a population of T cells in the cell processing medium into the internal view, or during the step of intermittently moving the base with respect to the top to enable transduction of the population of T cells;
vi) adding an additional volume of cell processing medium into the internal volume; and
vii) culturing the transduced population of T cells.

Advantageously, intermittent mixing of the contents of the compressible container during transduction of the T cells allows the cells to settle on the base during periods of rest, allowing for effective growth of the T cells, and also enables resuspension and dispersion of the T cells with the transduction agent during periods of mixing, to increase interaction between the T cells and the transduction agent for effective transduction of the T cells.

In examples, the step of culturing the transduced population of T cells comprises continuously moving the base with respect to the top, thereby continuously causing turbulence of contents in the internal volume, for a second period of time.

Advantageously, continuous mixing of the T cells during culturing of the cells creates turbulence in the cell solution comprised of the population of T cells in the cell processing medium to encourage oxygen held within the cell solution and within the headspace of the compressible container to mix with and permeate throughout the cell solution and thus increase the amount of dissolved oxygen in the cell solution.

According to a fifth aspect of the disclosure, there is provided a method of transducing and culturing T cells, comprising the ordered steps of:
i) providing a compressible container including a base, a top arranged substantially in parallel to the base, and at least one flexible wall element extending between the top and the base and defining an internal volume of the compressible container;
ii) adding a population of T cells in a cell processing medium into the internal volume;
iii) optionally maintaining the base stationary with respect to the top following the addition of the transduction agent for a predetermined period of time;
iv) rotating the base about an axis of rotation extending within a horizontal plane defined by the base, thereby causing turbulence of contents in the internal volume, for a first period of time;
v) maintaining the base stationary with respect to the top for a second period of time;
vi) repeating steps iv) and v) for a predetermined period of time;
vii) optionally adding additional cell processing medium into the internal volume;
viii) translating the base with respect to the top along a central longitudinal axis of the compressible container, thereby compressing the compressible container, for a third period of time; and
ix) adding a transduction agent into the internal volume during any of steps ii) to viii).

Advantageously, rotating the base for a first period of time, maintaining the base stationary for a second period of time and repeating such steps enables efficient mixing at lower volumes, in which transduction typically occurs, while translating the base for a third period of time enables efficient mixing at higher volumes, following addition of further cell processing medium required to maintain a desired growth profile of a cell culture.

The following examples are described in relation to any of the aspects of the disclosure (such as the first aspect, the second aspect, the third aspect, the fourth aspect or the fifth aspect) as described herein.

In examples, the method may further comprise moving the base with respect to the top, thereby causing turbulence of contents in the internal volume, for a second period of time. That is, rather than maintaining the base stationary for a second period of time, the base may be moved for the second period of time.

In examples, the first period of time may be 96 hours 72 hours, 48 hours, 24 hours, 12 hours, 6 hours, 3 hours, or 1 hour.

In examples, the second period of time may be 96 hours, 72 hours, 48 hours, 24 hours, 12 hours, 6 hours, 3 hours, or 1 hour. The step of moving the base with respect to the top for the first period of time may be the same, or different, duration of time as the step of moving the base with respect to the top for the second period of time.

In examples, the first period of time is 72 hours or 96 hours, and the second period of time is 24 hours.

In examples, the step of moving the base with respect to the top for the first period of time may comprise moving (as discussed below, for example, rotating) the base at a first rate, and the step of moving the base with respect to the top for the second period of time may comprise moving (as discussed below, for example, rotating) the base at a second rate. The first rate may be the same as, or different to, the second rate.

In examples, the second rate is greater than the first rate. This may be particularly advantageous in circumstances where a first volume of cell processing medium is provided within the internal volume during the step of moving the base at a first rate for a first period of time, and where a second volume of cell processing medium is provided within the internal volume during the step of moving the base at a second rate for a second period of time, where the second volume is greater than the first volume. In particular, appropriate mixing is achieved at two different cell processing media volumes without having to remove the contents from the internal volume.

In examples, the method may further comprise moving the base with respect to the top, thereby causing turbulence of contents in the internal volume, for a third period of time.

In such examples, the third period of time may be 96 hours 72 hours, 48 hours, 24 hours, 12 hours, 6 hours, 3 hours, or 1 hour.

In examples, the first period of time is 72 hours or 96 hours, the second period of time is 24 hours, and the third period of time is 72 hours or 96 hours.

In examples, the step of moving the base with respect to the top for the first period of time may comprise moving (as discussed below, for example, rotating) the base at a first rate, the step of moving the base with respect to the top for the second period of time may comprise moving (as discussed below, for example, rotating) the base at a second rate, and the step of moving the base for a third period of time may comprise moving (as discussed below, for example, compressing or longitudinally translating) the base at a third rate. The first, second and third rates may be the same or different.

In examples, the step of moving the base with respect to the top for the first period of time may comprise rotating the base (as discussed further below) at a first rate, the step of moving the base with respect to the top for the second period of time may comprise rotating the base (as discussed further below) at a second rate, and the step of moving the base for a third period of time may comprise longitudinally translating the base toward the top (as discussed further below) at a third rate. The first, second and third rates may be as defined further below.

In some examples, the first period of time may be from 1 minute to 5 days. The first period of time may be from 1 to 60 minutes. The first period of time may be from 0.5 hours (i.e., 30 minutes) to 5 hours. The first period of time may be 0.5 hours (i.e., 30 minutes). The first period of time may be from 1 to 3 hours. The first period of time may be 1 minute. The first period of time may be 3 hours. The first period of time may be 1 to 5 days. The first period of time may be 1 to 4 days. The first period of time may be 1 to 3 days.

In examples, the method may further comprise maintaining the base stationary with respect to the top for a second period of time.

In examples, the second period of time may be from 1 minute to 5 hours. The second period of time may be from 1 to 60 minutes. The second period of time may be from 0.5 to 5 hours. The second period of time may be from 1 to 3 hours. The second period of time may be 3 hours.

In examples, the steps of moving the base with respect to the top for the first period of time and maintaining the base stationary with respect to the top for the second period of time may be repeated. In some examples, such steps may be repeated a plurality (i.e., two or more) of times. In some examples, such steps are repeated continuously (such as successive moving and stationary periods) across 96 hours, 72 hours, 48 hours, or 24 hours.

In examples, the method may further comprise moving the base with respect to the top, thereby causing turbulence of contents in the internal volume, for a third period of time. Advantageously, this provides for intermittent mixing of the contents of the compressible container.

In examples, the third period of time may be 1 minute to 5 hours. The third period of time may be 1 to 60 minutes. The third period of time may be from 0.5 hours (i.e., 30 minutes) to 5 hours. The third period of time may be 0.5 hours (i.e., 30 minutes). The third period of time may be from 1 to 3 hours. The third period of time may be 1 minute. The third period of time may be 3 hours. The third period of time may be 1 to 5 days. The third period of time may be 1 to 4 days. The third period of time may be 1 to 3 days. In some examples, the third period of time is 4 days.

In examples, the step of moving the base with respect to the top for the first period of time may comprise rotating the base about an axis of rotation extending within a horizontal plane defined by the base.

In examples, the step of moving the base with respect to the top for the third period of time may comprise rotating the base about an axis of rotation extending within a horizontal plane defined by the base.

Advantageously, rotating the base about an axis of rotation within a horizontal plane of the base provides a rocking motion to agitate the contents of the compressible container. This increases the movement of the cells and the other contents within the cell processing medium to disperse the cells and increase interactions between the cells and the transduction agent. This can also create turbulence to encourage mixing of oxygen from the headspace of the compressible container or within the cell processing medium to thereby increase the amount of dissolved oxygen in the cell solution. Such a rocking motion may be particularly advantageous for mixing smaller volumes (such as less than or equal to 200 mL, or less than or equal to 150 mL, or less than or equal to 100 ml) of cell processing medium present in the container.

In examples, rotating the base may comprise rotating the base in a first direction about the axis of rotation into a first position in which the base forms a first angle with respect to the horizontal plane; and rotating the base in a second direction, opposite to the first direction, about the axis of rotation into a second position in which the base forms a second angle with respect to the horizontal plane.

In examples, the base may be rotated between the first position and the second position at a constant velocity.

In examples, the base may be rotated between the first position and the second position at a varying velocity.

In examples, the base may be maintained in the first position and/or the second position for a predetermined period of time.

In examples, the base may be rotated between the first position and the second position at a rate of 1 to 60 rotations per minute. The base may be rotated between the first position and the second position at a rate of 1 to 30 rotations per minute. The base may be rotated between the first position and the second position at a rate of 5 to 25 rotations per minute. The base may be rotated between the first position and the second position at a rate of 10 to 20 rotations per minute. The base may be rotated between the first position and the second position at a rate of about 5, 10, 20, 30, 40, 50, or 60 rotations per minute. Such rate of rotations may be regarded as the rate for the first rate, second rate or third rate as discussed herein.

In particular examples, the base may be rocked. In this sense, the base may be rotated from the first position, to the second position, and back to the first position. The base may be rocked at a rate of 1 to 60 rocks per minute, 1 to 30 rocks per minute, 5 to 25 rocks per minute, or 10 to 20 rocks per minute. Preferably, the base is rocked at a rate of 10 rocks per minute for 0.5 hours (i.e., 30 minutes). Herein, a rock is defined as a rotation from the first position, to the second position, and back to the first position. Such rate of rocking may be regarded as the rate for the first rate, second rate or third rate as discussed herein.

In examples, the first angle and/or the second angle may be 1 to 45 degrees. The first angle and/or the second angle may be 5 to 25 degrees. The first angle and/or the second angle may be about 5 degrees, 10 degrees, 15 degrees, 20 degrees, or 25 degrees. In examples, the second angle may be equal to the first angle. Preferably, the first angle and/or the second angle, and most preferably each of the first angle and second angle, is about 5 degrees, about 10 degrees or about 15 degrees.

In examples, moving the base with respect to the top for the first period of time may comprise translating the base toward the top along a central longitudinal axis of the compressible container, thereby compressing the compressible container.

In examples, moving the base with respect to the top for the third period of time may comprise translating the base toward the top along a central longitudinal axis of the compressible container, thereby compressing the compressible container.

Advantageously, moving the base along a central longitudinal axis of the compressible container creates a reciprocal compression motion to agitate the contents of the compressible container. This increases the movement of the cells and the other contents of the compressible container. This also creates turbulence to encourage mixing of oxygen in the headspace of the compressible container with the contents in the compressible container and increase the amount of dissolved oxygen in the cell solution. Such a compression motion may be particularly advantageous for mixing larger volumes (such as greater than or equal to 200 mL, or greater than or equal to 150 mL, or greater than or equal to 100 ml) of cell processing medium present in the container.

In examples, the compressible container may be compressed at a rate of 1 to 60 compressions per minute. The compressible container may be compressed at a rate of 1 to 30 compressions per minute. The compressible container may be compressed at a rate of 5 to 25 compressions per minute. The compressible container may be compressed at a rate of 10 to 20 compressions per minute. The compressible container may be compressed at a rate of about 5, 10, 20, 30, 40, 50, or 60 compressions per minute. Herein, compressions per minute is defined as translation of the base toward the top, from a first position to a second position, along the central longitudinal axis of the compressible container, and then translation of the base away from the top, from the second position back to the first position. Such rate of compressions may be regarded as the rate for the first rate, second rate or third rate as discussed herein.

In examples, moving the base with respect to the top for the first period of time may comprise pivoting the base about a centrally disposed origin of the base.

In examples, moving the base with respect to the top for the third period of time may comprise pivoting the base about a centrally disposed origin of the base.

Advantageously, pivoting the base about the central origin of the base provides a pivoting motion to agitate the contents of the compressible container. This increases the movement of the cells and the other contents within the cell processing medium to disperse the cells and increase interactions between the cells and the transduction agent. This can also create turbulence to encourage mixing of oxygen from the headspace of the compressible container or within the cell processing medium and increase the amount of dissolved oxygen in the cell solution. Such a pivoting motion may be particularly advantageous for mixing smaller volumes (such as less than or equal to 200 mL, or less than or equal to 150 mL, or less than or equal to 100 ml) of cell processing medium present in the container.

In examples, the base may be pivoted at a rate of 1 to 60 revolutions per minute. The base may be pivoted at a rate of 1 to 30 revolutions per minute. The base may be pivoted at a rate of 5 to 25 revolutions per minute. The base may be pivoted at a rate of 10 to 20 revolutions per minute. The base may be pivoted at a rate of about 5, 10, 20, 30, 40, 50, or 60 revolutions per minute. Herein, a revolution is defined as rotating, or pivoting, the base circumferentially about the central origin in either a clockwise or anticlockwise direction, starting at a first position and finishing at that same first position. Such rate of pivoting may be regarded as the rate for the first rate, second rate or third rate as discussed herein.

In examples, the method further comprises the step of moving the base with respect to the top for an additional predetermined, such as a fourth, period of time. Such step of moving the base with respect to the top for the additional predetermined, such as the fourth, period of time may comprise any one or more steps of rotating, rocking, translating (i.e., compressing) or pivoting the base as contemplated herein. Any of the previous moving steps may form the additional predetermined, such as the fourth, period of time. The additional predetermined, such as the fourth, period of time may be repeated one or more times.

In examples, the method comprises (a) moving the base with respect to the top for a first period of time, (b) maintaining the base stationary with respect to the top for a second period of time, repeating steps (a) and (b) for a predetermined period of time, and (c) moving the base with respect to the top for a third period of time. In preferred examples, step (a) comprises rotating (or rocking) the base about an axis of rotation extending within a horizontal plane defined by the base. In preferred examples, step (c) comprises translating the base toward the top along a central longitudinal axis of the compressible container, thereby compressing the compressible container. In such preferred examples, the first period of time may be 0.5 hours (i.e., 30 minutes). In such preferred examples, the second period of time may be 3 hours. In such preferred examples, steps (a) and (b) may be repeated for 72 hours, 48 hours or 24 hours, most preferably 72 hours. In such preferred examples, the third period of time may be 96 hours, 72 hours, 48 hours or 24 hours, most preferably 96 hours.

In examples, the transduction agent may be added into the internal volume during the step of moving the base with respect to the top for the first period of time.

In examples, the transduction agent may be added into the internal volume during the step of moving the base with respect to the top for the third period of time.

In examples, the transduction agent may be a viral vector. The viral vector may be a lentiviral vector. The lentiviral vector may be a CD19 CAR lentiviral vector (LVV). The lentiviral vector may be a green fluorescent protein (GFP) lentiviral vector (LVV).

In examples, the flexible wall element may comprise a wall having one or more folds, such as Z-folds. Advantageously, the wall of the compressible container is easily manipulated for compression and expansion of the compressible container. In examples, the base (and/or the top) may be compressible (i.e., configured to be translated toward or away) from the top (and/or the base).

In examples, the compressible container may be substantially gas-impermeable. Advantageously, the movement of the base with respect to the top causes turbulence of contents in the internal volume of the compressible container, which mixes oxygen in the headspace of the compressible container with the cell processing medium, increasing the levels of dissolved oxygen within the cell processing medium. As such, sufficient oxygenation of the cell processing medium can be achieved in a non-gas-permeable (i.e., gas impermeable) container.

In examples, the compressible container may be at least partially gas-permeable.

In examples, the base and/or the flexible wall element may comprise a gas-permeable material, such as silicone or fluorinated ethylene propylene.

In examples, the compressible container may comprise a mixing element, such as a baffle, a static impeller or the like, within the internal volume. In other examples, the compressible container is provided without any mixing element within the internal volume.

In examples, the T cells may be selected from CD4+ T cells and CD8+ T cells. In examples, the T cells may be CD3+ T cells or CD34+ hematopoietic stem/progenitor cells (HSPCs). In examples, the T cells may be CD3+ T cells having CD4+ markers. In examples, the T cells may be CD3+ T cells having CD8+ markers. In examples, the T cells may comprise CD3+ T cells having CD4+ markers and CD3+ T cells having CD8+ markers.

In examples, the method may further comprising adding an activation agent into the internal volume. Adding the activation agent into the internal volume may be at the same time as adding the population of T cells in the cell processing medium into the internal volume.

In examples, for example, in respect of the third aspect, the predetermined period of time for repeating steps iv) and v) may be 1 to 5 days. The predetermined period of time may be 2 to 3 days. The predetermined period of time may be about 3 days. Such examples are not limited to the third aspect and are equally applicable to other aspects as disclosed herein.

In examples, the step of adding the transduction agent into the internal volume may occur during the step of adding the population of T cells in the cell processing medium into the internal volume.

In examples, the step of adding the transduction agent into the internal volume may occur during the step of moving the base with respect to the top for the first period of time.

In examples, the method further comprises the step of maintaining the base stationary with respect to the top following the addition of the transduction agent for a predetermined period of time.

In examples, the predetermined period of time may be 1 day (i.e., 24 hours), 12 hours, 6 hours, 3 hours, 2 hours or 1 hour.

In examples, the step of adding the transduction agent into the internal volume may occur during the step of maintaining the base stationary with respect to the top for the second period of time.

In examples, the step of adding the transduction agent into the internal volume may occur during the repetition of either of the steps of moving the base with respect to the top for the first period of time or maintaining the base stationary with respect to the top for the second period of time.

In examples, the step of adding a transduction agent into the internal volume may occur during the step of intermittently moving the base with respect to the top for the first period of time.

In examples, for example, in respect of the fourth aspect, intermittently moving the base with respect to the top for the first period of time and continuously moving the base with respect to the top for the second period of time may comprise moving the base with respect to the top according to a first motion. Such examples are not limited to the fourth aspect and are equally applicable to other aspects as disclosed herein.

In examples, the first motion may comprise rotating the base about an axis of rotation extending within a horizontal plane defined by the base. Advantageously, rotating the base about an axis of rotation within a horizontal plane of the base provides a rocking motion to agitate the contents of the compressible container. This increases the movement of the cells and the other contents of the compressible container to disperse the cells and increase interactions between the cells and the transduction agent. This can also create turbulence to encourage mixing from oxygen in the headspace of the compressible container or within the cell processing medium and increase the amount of dissolved oxygen in the cell processing medium. This rocking motion is suitable for mixing low volumes of cell processing medium in the internal volume of the compressible container.

In examples, for example, in respect of the third fourth, the method of transducing and culturing T cells may further comprise the steps of: viii) adding an additional volume of cell processing medium into the internal volume; and ix) culturing the population of T cells while continuously moving the base with respect to the top according to a second motion, thereby continuously causing turbulence of contents in the internal volume, for a third period of time. Such examples are not limited to the fourth aspect and are equally applicable to other aspects as disclosed herein.

In examples, the second motion may comprises translating the base toward the top along a central longitudinal axis of the compressible container, thereby compressing the compressible container. Advantageously, moving the base along a central longitudinal axis of the compressible container creates a reciprocal compression motion to agitate the contents of the compressible container. This increases the movement of the cells and the other contents of the compressible container. This also creates turbulence to encourage mixing of oxygen from the headspace of the compressible container or within the cell processing medium and increase the amount of dissolved oxygen in the cell processing medium. This reciprocal compression motion is suitable for mixing large volumes of cell solution in the internal volume of the compressible container.

In examples, the step of adding a population of T cells in a cell processing medium into the internal volume comprises adding a population of T cells in a first volume of cell processing medium into the internal volume. The first volume may be 200 mL, 150 mL, 100 mL, 50 mL, 25 mL or 10 mL. The first volume may be less than or equal to 200 mL, less than or equal to 150 mL, less than or equal to 100 mL, less than or equal to 50 mL, less than or equal to 25 mL or less than or equal to 10 mL.

In examples, following the addition of a transduction agent into the internal volume, a second volume of cell processing medium is added to the internal volume. The second volume of cell processing medium may be equal to, or greater than, the first volume of cell processing medium. Subsequent additions of a volume (e.g., a third volume, a fourth volume etc.) of cell processing medium are equally contemplated, and may be equal to, or greater than, the first volume or the second volume. In some examples, the second volume may be 200 ml, 150 mL, 100 mL, 50 mL, 25 mL or 10 mL.

In examples, following the addition of a transduction agent into the volume, one or more additional volumes of cell processing medium is or are added to the internal volume. The additional volume(s) of cell processing medium may be equal to the volume of cell processing medium present in the internal volume prior to the addition of such additional volume(s).

### BRIEF DESCRIPTION OF THE DRAWINGS

FIG. 1 illustrates a cross-sectional view of a bioreactor according to the present disclosure.

FIG. 2 illustrates a cross-sectional view of another bioreactor according to the present disclosure.

FIG. 3 illustrates a perspective view of an agitator for engaging a base of the bioreactor of FIG. 1 or FIG. 2.

FIGS. 4A-4D illustrate a front view of an agitator plate (FIG. 4A) in a lower position, (FIG. 4B) in a higher position, (FIG. 4C) in a first angular position, and (FIG. 4D) in a second angular position.

FIG. 5 illustrates a flow chart of a “quick mixing” method of transducing cells according to the present disclosure.

FIG. 6 illustrates a flow chart of an “intermittent mixing” method of transducing cells according to the present disclosure.

FIG. 7 illustrates a flow chart of a “continuous mixing” method of transducing cells according to the present disclosure.

FIG. 8 illustrates a line graph of the total number of viable cells in a compressible container according to the present disclosure, a rigid gas-permeable container, and a gas-permeable bag over a seven day period.

FIGS. 9A and 9B illustrate a box graph of the transduction efficiencies in a compressible container according to the present disclosure, a rigid gas-permeable container, a gas-permeable bag, and a rigid container with a stirrer (FIG. 9A) for CD8+ T cells and (FIG. 9B) for CD4+ T cells.

FIG. 10 illustrates various examples of mixing modes for the compressible container according to the present disclosure, and a rigid gas-permeable container control, across days 0 to 3 of a culture process.

FIG. 11 illustrates a comparison of cell growth according to various mixing modes for the compressible container according to the present disclosure, and according to a static mode of a rigid gas-permeable container control.

FIG. 12 illustrates a comparison of transduced cell yield according to various mixing modes for the compressible container according to the present disclosure, and according to a static mode of a rigid gas-permeable container control.

FIG. 13 illustrates a comparison of transduction efficiency according to various mixing modes for the compressible container according to the present disclosure, and according to a static mode of a rigid gas-permeable container control.

### DETAILED DESCRIPTION

The described example embodiments relate to a method of transducing T cells within a compressible container. As will be recognized by a person skilled in the art, references to container, receptacle, bioreactor or the like are intended to be used synonymously in the detailed description are not intended to limit the scope of protection. As will be recognized by a person skilled in the art, the containers described herein are mere examples of suitable containers.

Certain terminology is used in the following description for convenience only and is not limiting. The words “upper” or “top,” and “lower” or “bottom,” designate directions in the drawings to which reference is made and are with respect to the described component when assembled. Similarly, the words “inner” or “inwardly,” and “outer” or “outwardly,” refer to directions toward and away from, respectively, a designated centerline or a geometric center of an element being described (e.g., a central axis), the particular meaning being readily apparent from the context of the description. Further, the terms “proximal” (i.e., nearer to) and “distal” (i.e., away from) designate positions relative to an axis or a point of attachment.

Further, as used herein, the terms “connected,” “affixed,” “coupled,” and the like are intended to include direct connections between two members without any other members interposed therebetween, as well as indirect connections between members in which one or more other members are interposed therebetween. The terminology includes the words specifically mentioned above, derivatives thereof, and words of similar import.

Further, unless otherwise specified, the use of ordinal adjectives, such as, “first,” “second,” “third,” etc., merely indicate that different instances of like objects or terms and are not intended to imply that the objects so described must be in a given sequence, either temporally, spatially, in respect of a methodology, in ranking or in any other manner. Like reference numerals are used to depict like features throughout.

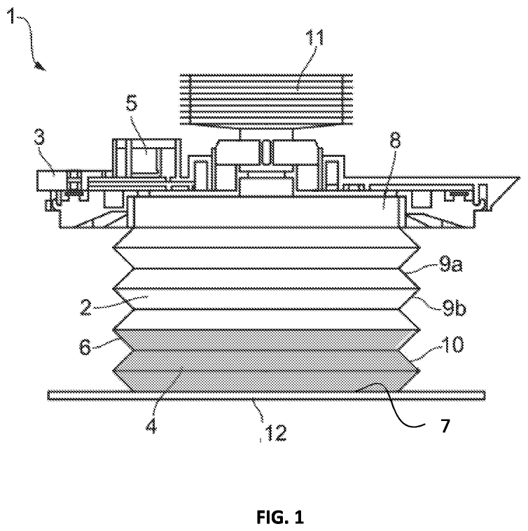

The bioreactor 1 shown in FIG. 1 includes a cell culture container 2 and an interface plate 3. During use, the cell culture container 2 holds a fluid in which the cell processing occurs. In particular, the fluid is a cell suspension 4 comprising a population of cells, for example, T cells, present in a cell processing medium. Additionally, a headspace is present within the cell culture container 2, above the upper surface of the cell processing medium. The headspace fills the remaining volume of the bioreactor not otherwise occupied by cell processing medium. The cell suspension may also comprise a transduction agent. The cells are transduced in the cell culture container by the introduced transduction agent to produce genetically modified cells. The cells are also cultured in the cell culture container 2 to reproduce, and may be otherwise treated, to create a cell-based therapeutic product.

The interface plate 3 is attached to a top of the cell culture container 2 thereby acting as a lid or closure. The interface plate 3 comprises at least one connector interface 5 for connecting to an external component, for example, an external container configured to deliver a fluid to, or configured to extract a fluid from, the cell culture container 2. In some examples, the interface plate 3 includes a plurality of connector interfaces 5, each for connecting to one of a plurality of external components. Each connector interface 5 may be used once or more for adding or removing fluid. The connector interfaces 5 may be distributed about the interface plate 3. Accordingly, the interface plate 3 provides for adding cell processing medium and other fluids to the cell culture container 2 during cell processing, and/or for removing fluid from the cell culture container 2 during processing, for example, to remove a sample or waste fluid.

An external container (not shown) may be connectable to the connector interface 5 in the interface plate 3 to add material to, or extract material from, the cell culture container 2. The external container may contain a material for addition into the cell culture container 2 during cell processing. For example, the external container may contain a cell processing medium, transduction agents, activation agents, cytokines, growth factors, magnetic beads, or the like.

The cell culture container 2 is a compressible container. Accordingly, the cell culture container is flexible such that it is extendible and compressible. In particular, the cell culture container 2 has a compressible wall element 6, for example, a bellows wall. The cell culture container 2 has a base 7 disposed opposite to the interface plate 3, and a compressible wall element 6 defining a sidewall of the cell culture container 2. A top part of the compressible wall element 6 is attached to the interface plate 3. The top part of the compressible wall element 6 may include a rigid ring 8 or similar for attaching to the interface plate 3. The rigid ring 8 is threaded to engage with corresponding threads of the interface plate 3 for coupling thereto. The compressible wall element 6 is compressible and/or extendible such that the base 7 can move toward and away from the interface plate 3, changing the internal volume of the cell culture container 2. The base 7 may be moved relative to the interface plate 3 in order to agitate or mix the cell suspension 4 in the cell culture container 2.

The compressible wall element 6 may be a bellows wall, having a concertina arrangement that allows the compressible wall element 6 to fold onto itself in order to compress. In particular, as illustrated the compressible wall element 6 may comprise a series of alternately arranged deformable portions 9a, 9b. Leaf segments 10 extend between, i.e., interleave, the deformable portions 9a, 9b. The leaf segments 10 are more rigid than the deformable portions 9a, 9b. The deformable portions 9a, 9b act as hinges that allow the compressible wall element 6 to collapse like a bellows or concertina, with the leaf segments 10 remaining substantially non-deformed.

The compressible wall element 6 may comprise at least one inwardly deformable portion 9a and at least one outwardly deformable portion 9b, for example, at least two inwardly deformable portions 9a and at least two outwardly deformable portions 9b. The compressible wall element 6 may comprise three, four, or more inwardly deformable portions 9a and three, four or more outwardly deformable portions 9b.

The inwardly deformable portion(s) 9a and outwardly deformable portion(s) 9b may be formed by thinned sections in the compressible wall element 6. The inwardly deformable portion(s) 9a may comprise a thinned section arranged on the outer surface of the compressible wall element 6 such that it is deformable in an inwards direction. The outwardly deformable portion(s) 9b may comprise a thinned section arranged on the inner surface of the compressible wall element 6 such that it is deformable in an outwards direction.

The compressible wall element 6 may be formed from a gas permeable or a gas-impermeable material. In examples, the compressible wall element 6 comprises a silicone, in particular, a liquid silicone rubber, or fluorinated ethylene propylene (FEP). In other examples, the compressible wall element 6 comprises a low density polyethylene (LDPE). In other examples, the compressible wall element 6 comprises a thermoplastic elastomer (TPE), such as a gas-impermeable TPE. In examples, as described further hereinafter, the compressible wall element 6 may be coated, laminated, or otherwise treated to reduce the gas permeability of the compressible wall element 6 or to render the compressible wall element 6 impermeable to gases, particularly oxygen. In some examples, the compressible wall element 6 comprises an inner portion and an outer sheath, jacket, or coating. For example, the compressible wall element 6 may comprise an inner portion and a jacket over-molded onto the inner portion. The inner portion may comprise LDPE and the jacket may comprise a TPE. In another example, the compressible wall element 6 may comprise an elastomer outer sheath, for example, a TPE outer sheath, and a liner. The sheath may assume the form of a container and the general shape of cell culture container 2. For example, an LDPE liner may be blow-mounted onto the internal surface of the TPE outer sheath to form the liner. In another example, the liner may be an insert, for example, an LDPE insert, received within the elastomer outer but not co-molded with the TPE outer sheath. In such an example it may be preferable that the liner comprises a base sheet and defines a sealed container (except for the top) to hold the cell suspension 4.

The cell culture container 2 can therefore expand and contract, or be expanded and contracted, according to the material held in the cell culture container 2. In particular, the cell culture container 2 may expand as the volume of cell suspension 4 within the cell culture container 2 grows, and/or as additional materials are added.

As illustrated, the interface plate 3 also includes an expansion container 11. The expansion container 11 allows for the cell culture container 2 to expand and contract without greatly changing the pressure in the cell culture container 2. Alternatively or additionally, the expansion container 11 may be operable, for example, by being mechanically or manually compressed or expanded, to expand or retract the compressible wall 6 element of the cell culture container 2 and thereby change a volume of the cell culture container 2. Alternatively or additionally, the expansion container 11 may be operable, for example, by being mechanically or manually compressed or expanded, to alter the pressure within the cell culture container 2.

In various examples, the cell culture container 2 includes the base 7 coupled thereto. The base 7 is generally planar, i.e., flat, and rigid. The base 7 is attached to, or co-molded with, the compressible wall element 6.

The base 7 is substantially planar and thereby defines a rigid, substantially flat bottom of the cell culture container 2. A flat base 7 of the cell culture container 2 may provide for improved cell culturing, in particular, mixing and control over cell culturing. The base 7 of the cell culture container 2 thus helps to ensure that cells are substantially evenly spread over the cross-section of the cell culture container 2 as the cells settle on the bottom of the cell culture container 2. This mitigates cells “piling up” in isolation positions on the base 7, which may lead to depleted oxygen levels in such isolated positions. The flat base 7 of the cell culture container 2 also helps to prevent cell suspension 4 being trapped in the cell culture container 2 when the cells are harvested or extracted at the end of the cell culturing process.

In various examples, the base 7 comprises a thermoplastic, for example, a high density polyethylene (HDPE), or a polycarbonate (PC), or another rigid polymer.

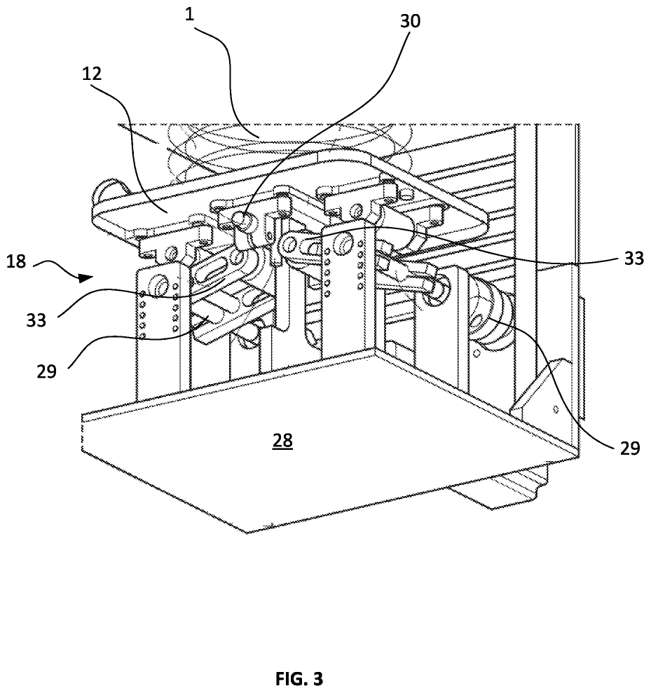

Additionally, as shown in FIG. 1, an agitator plate 12 is provided in abutting engagement with the base 7. The agitator plate 12 is described in more detail in relation to FIG. 3. The agitator plate 12 may be coupled, e.g., directly coupled, to the base 7 in some examples.

In the illustrated examples the cell culture container 2 is generally cylindrical, with a generally circular base 7 and a generally cylindrical compressible wall element 6. Accordingly, an axial direction is defined between the base 7 and the end of the compressible wall element 6 where the interface plate 3 is mounted. However, it will be appreciated that the cell culture container 2 may take an alternative form, such as having a generally triangular or square cross-sectional form.

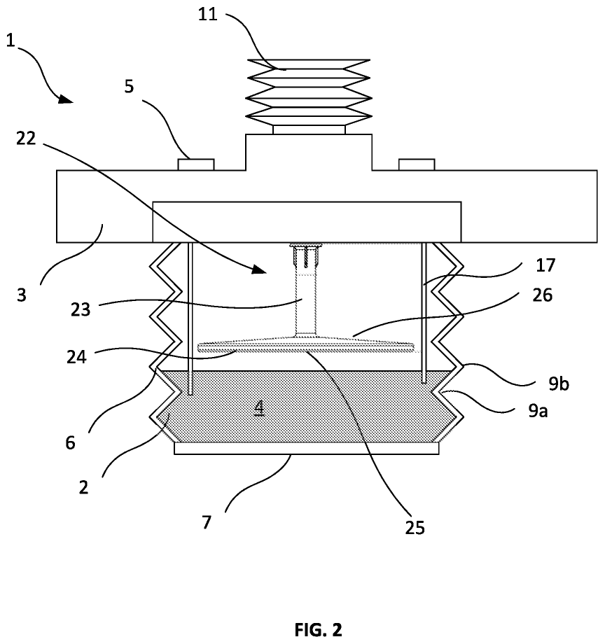

As shown in FIG. 2, which shows a cross-section of another example of the bioreactor 1 according to the present disclosure, the bioreactor 1 includes a mixing element such as a baffle 22. The baffle 22 is mounted to the interface plate 3 such that the baffle 22 is suspended within the cell culture container 2. The baffle 22 includes a mounting portion 23 that is attachable to the interface plate 3. In examples, the mounting portion 23 is attachable to the interface plate 3 by a threaded connector, or by a clip or clamp. In the illustrated examples, the mounting portion 23 is attached to the center of the interface plate 3 such that the baffle 22 is centrally positioned within the cell culture container 2. However, it will be appreciated that the baffle 22 may be positioned off-center within the cell culture container 2. The mounting portion 23 extends from the interface plate 3 toward the base 7, and a baffle member 24 is attached to the mounting portion 23 or formed therewith. In this example, the baffle member 24 comprises a substantially flat bottom surface 25 facing the base 7 of the cell culture container 2. The baffle member 24 also has a conical upper surface 26, facing the interface plate 3, though this may also be substantially flat in alternative examples.

The baffle member 24 is circular and is sized so as to fit within the cell culture container 2. In examples, the baffle member 24 is sized so as to be spaced from the compressible wall element 6 of the cell culture container 2. The baffle member 24 may be sized so as to be spaced from the compressible wall element 6 of the cell culture container 2 by permitting a sampling tube 17 to pass between the compressible wall element 6 and the baffle member 24. The sampling tube 17 provides a fluid sampling path from the cell culture container 2 to the interface plate 3. In examples, the baffle member 24 may be spaced from the compressible wall element 6 by a distance of between about 5 millimeters and about 20 millimeters.

The baffle 22 is provided to mix contents of the bioreactor 1 during use. In particular, the base 7 of the bioreactor 1 may be moved relative to the interface plate 3 and baffle 22 such that the baffle 22 contacts the cell suspension 4 within the cell culture container 2 and mixes it. In examples, the base 7 may be raised and lowered relative to the interface plate 3 (i.e., to change a distance between the base 7 and the interface plate 3), and/or the base 7 may be tilted relative to the interface plate 3, and/or the base 7 may be rotated relative to the interface plate 3, as will be described in further detail below.

After mixing, the baffle 22 may be removed from the cell suspension 4, via expansion of the cell culture container 2, and the conical upper surface 26 of the baffle member 24 ensures that fluid is not retained on the baffle 22 and instead runs back into the cell culture container 2.

As shown in FIG. 3, the bioreactor 1 is positioned on an agitator 18. The agitator 18 includes an agitator plate 12 that engages the base 7 of the bioreactor 1 to move the base 7 relative to the interface plate 3 and thereby agitate the contents of the bioreactor 1. Such agitation mixes the fluids of the bioreactor 1 to help the transduction process. The agitation can also help the cell culturing process, for example, by mixing fluids within the bioreactor 1, or by encouraging oxygen to dissolve into the cell culture. The agitator 18 may be mounted within an incubator housing (not shown).

FIG. 3 illustrates an example agitator 18 with an actuator mechanism arranged to move an agitator plate 12 relative to the bioreactor 1. As shown, the agitator plate 12 is moveable to engage the bioreactor 1, in particular, the base (i.e., base 7, see FIG. 1). The actuator mechanism is mounted on an agitator base plate 28. Between the agitator base plate 28 and the agitator plate 12 is one or more actuators 29 that act to raise and lower the agitator plate 12.

In the illustrated example, the actuators 29 are motors arranged to rotate articulated crank arms 33 that are rotatably connected to the agitator base plate 28 and to the agitator plate 12 such that rotation of the articulated crank arms 33 moves the agitator plate 12. In other examples, the linear actuators may be provided to act directly between the agitator base plate 28 and the agitator plate 12.

Supports and guides may guide the movement of the agitator plate 12.

The actuator mechanism may further include a pivotable rod 30 such that the agitator plate 12 can pivot about the pivotable rod 30 to tilt the base of the bioreactor 1. The actuators 29 enable pivoting of the agitator plate 12 about the pivotable rod 30 to tilt the base of the bioreactor 1. Accordingly, the agitator plate 12 can be moved relative to the agitator base plate 28 in order to engage the base of the bioreactor 1 and agitate the contents of the bioreactor 1.

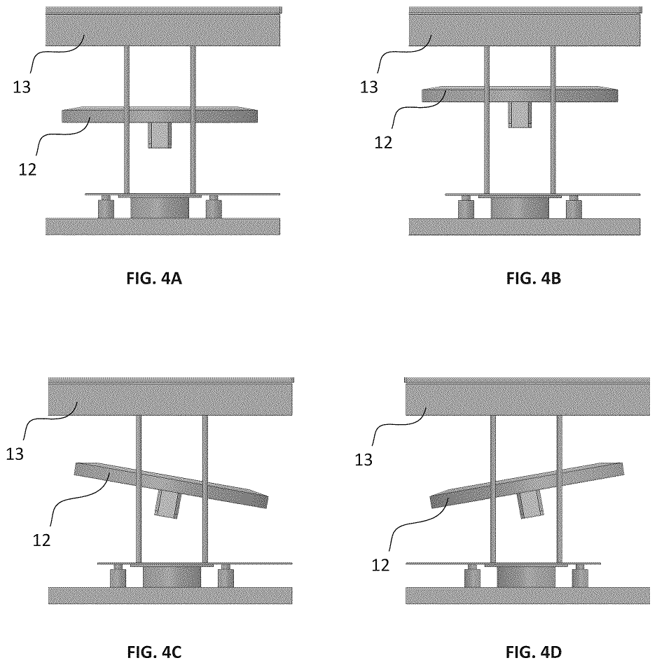

FIGS. 4A to 4D illustrate agitative movements of the agitator plate 12 without the bioreactor 1 or actuator mechanism shown. FIGS. 4A to 4D show a receiving portion 13, which supports, holds, and clamps the interface plate 3 of the bioreactor 1 during use, with the cell culture container (i.e., cell culture container 2, see FIG. 1) suspended below the receiving portion 13. The receiving portion 13 holds the interface plate 3 in a horizontal position such that actuation of the base 7 by the agitator plate 12 moves the base 7 relative to the interface plate 3.

As shown in FIGS. 4A and 4B, the agitator plate 12 is translated between lower and higher positions along a central longitudinal axis of the cell culture container 2. This movement of the agitator plate 12 between the lower and higher positions agitates the contents in the internal volume of the cell culture container 2. The movement between the lower and higher positions may be a reciprocal compression motion to provide agitation or turbulence of the contents of the cell culture container 2 for a predetermined period of time, as will be discussed in further detail below. The compression motion allows for mixing of large volumes of cell suspension 4 in the cell culture container 2. In some examples, the baffle 22 increases mixing of the cell suspension 4 during compression mixing.

The compression motion may be at a rate of 1 to 60 cycles per minute (cpm), each cycle being a compression (i.e., translation of the base 7 toward the interface plate 3) and a retraction (i.e., translation of the base 7 away from the interface plate 3) of the cell culture container 2. In one example, the compression motion may be at a rate of 1 to 30 cpm. In another example, the compression motion may be at a rate of 5 to 25 cpm. In another example, the compression motion may be at a rate of 10 to 20 cpm. In other examples, the compression motion may be at a rate of 5 cpm, 10 cpm, 20 cpm, 30 cpm, 40 cpm, 50 cpm, or 60 cpm.

As shown in FIGS. 4C and 4D, the agitator plate 12 is tilted to agitate the contents of the bioreactor 1. In FIG. 4C, the agitator plate 12 is tilted in a first position in which the base forms a first angle with respect to the horizontal plane. The agitator plate can be rotated in an opposite direction to a second position, as shown in FIG. 4D, in which the base forms a second angle with respect to the horizontal plane. The first angle and the second angle are between, and including, 1 degree and 45 degrees. Preferably, the first angle and the second angle are between, and including, 5 to 25 degrees. The first angle and the second angle may be one of 5 degrees, 10 degrees, 15 degrees, 20 degrees and 25 degrees. The second angle may be equal to the first angle. Movement of the agitator plate 12 between the first position and the second position creates a rocking motion. This rocking motion gently agitates, and causes turbulence of, the cell suspension 4 in the cell culture container 2. The motion of the agitator plate 12 between the first and second positions may have a varying velocity. Alternatively, the motion of the agitator plate 12 between the first and second positions may have a constant velocity. In examples utilizing a constant velocity, the base may be held at each of the first angle and the second angle for a predetermined period of time, such as from 0.1 to 5 seconds. The movement between the first and second positions may be a reciprocal motion to provide agitation of the contents of the bioreactor 1 for a predetermined period of time, as will be discussed in further detail below.

The rocking motion may be at a rate of 1 to 60 rocks per minute (rpm), each rock being a rotation starting from a first position (e.g., FIG. 4C), rotating to a second position (e.g., FIG. 4D), and then rotating back to, and returning to, the first position (e.g., FIG. 4C). In one example, the rocking motion may be at a rate of 1 to 30 rpm. In another example, the rocking motion may be at a rate of 5 to 25 rpm. In another example, the rocking motion may be at a rate of 10 to 20 rpm. In other examples, the rocking motion may be at a rate of 5 rpm, 10 rpm, 20 rpm, 30 rpm, 40 rpm, 50 rpm, or 60 rpm.

A combination of vertical and tilting movements may be provided to agitate the contents of the bioreactor 1. The agitator plate 12 may be tiltable in different directions, and/or the agitator plate 12 may only be tiltable in one or two directions but rotation of the bioreactor 1 can change the direction of tilt of the bioreactor 1 itself. This provides a pivotal motion about a central point of the base 7 of the bioreactor 1.

In a further example, not illustrated, the base 7 may be pivoted about a centrally disposed origin of the base. This creates a swirling motion to agitate the contents in the internal volume of the cell culture container 2. The base may be pivoted to a first angle relative to the horizontal plane. The first angle is between, and including, 1 degree and 45 degrees. Preferably, the first angle is between, and including, 5 to 25 degrees. The first angle may be one of 5 degrees, 10 degrees, 15 degrees, 20 degrees and 25 degrees.

The base may be pivoted at a rate of 1 to 60 revolutions per minute, each revolution being one circumferential rotation about the origin. In one example, the pivoting motion may be at a rate of 1 to 30 revolutions per minute. In another example, the pivoting motion may be at a rate of 5 to 25 revolutions per minute. In another example, the pivoting motion may be at a rate of 10 to 20 revolutions per minute. In other examples, the pivoting motion may be at a rate of 5 revolutions per minute, 10 revolutions per minute, 20 revolutions per minute, 30 revolutions per minute, 40 revolutions per minute, 50 revolutions per minute, or 60 revolutions per minute.

In some examples, the agitator plate 12 may be coupled to the base 7 of the bioreactor 1 such that the base 7 will move with the agitator plate 12. In other examples, the agitator plate 12 may not couple to the base 7, the base 7 may lift wholly or partially away from the agitator plate 12 in some positions and/or during some agitative movements. Some agitative movements may provide impact contacts, i.e., engagement, between the agitator plate 12 and the base 7, in order to agitate the contents of the bioreactor. In other examples, the agitator plate 12 may be a vibrating agitator plate configured to vibrate the base 7 of the bioreactor 1.

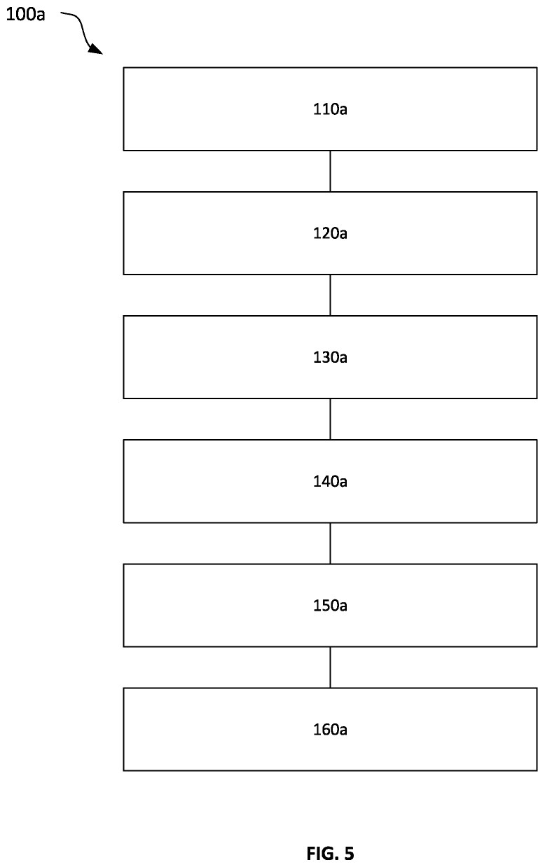

A method of transducing cells using the bioreactor 1 as described above will now be described with reference to FIGS. 5 to 7. Prior to starting the transduction method, target cells are isolated from a patient or donor sample. Any suitable target cells requiring transduction may be used. In some examples, the target cells may be T cells, such as CD3+, CD4+, or CD8+ T cells. In other examples, the target cells may be CD34+ hematopoietic stem and progenitor cells (HSPCs).

In each method 100a, 100b, 100c, T cells are added to an internal volume of the cell culture container (i.e., cell culture container 2, see FIGS. 1 and 2) in a cell processing medium at step 110a, 110b, 110c. In other words, inoculation of T cells into the cell culture container occurs on day 0.

The cell processing medium may be selected from any suitable medium. In some examples, the cell processing medium may be Dulbecco's Modified Eagle Medium (DMEM), available from Thermo Fisher Scientific, Sigma Aldrich and others, X-VIVO™ 15, available from Lonza, or TexMACS™, available from Miltenyi Biotec. The cell processing medium may be any cell processing medium suitable for T cells.

Optionally, an activation agent may be added to the cell suspension 4 in the cell culture container (i.e., cell culture container 2, see FIGS. 1 and 2). The activation agent may be added in the internal volume of the cell culture container at the same time as the T cells (i.e., on day 0). The activation agent may be one of magnetic beads or soluble particles. The contents including the activation agent-of the cell culture container may be agitated according to one of the mixing methods outlined below (at steps 130a and 140a, steps 130b, 140b and 145b, or at steps 130c and 140c). Agitation allows for mixing of the activation agent with the cell processing medium.

A transduction agent is added to the internal volume of the cell culture container (i.e., cell culture container 2, see FIGS. 1 and 2) at step 120a, 120b, 120c. The transduction agent may be a viral vector. Preferably, the viral vector is a lentiviral vector. The transduction agent may be added from 6 hours to 2 days after addition of the activation agent. Preferably, the transduction agent is added 1 day (24 hours) after addition of T cells and the activation the activation agent (i.e., the transduction agent is added on day 1).

The addition of transduction agent, i.e., step 120a, 120b, 120c, may be prior to, during, or after any of steps 130a, 130b or 130c, steps 140a, 140b or 140c, or step 145b, as discussed in further detail below.

The cell suspension 4 including the cells, cell processing medium and transduction agent can be mixed in the cell culture container by moving the base(i.e., base 7, see FIGS. 1 and 2) of the cell culture container according to a “quick mixing” method, an “intermittent mixing” method, and/or a “continuous mixing” method. This mixing agitates the cell suspension to stimulate transduction of the cells in the cell suspension.

The “quick mixing” method is shown in FIG. 5. The base (i.e., base 7, see FIGS. 1 and 2) of the cell culture container is moved with respect to the top (i.e., interface plate 3, see FIGS. 1 and 2) for a first period of time at step 130a. The base may be caused to move by the agitator (i.e., agitator 18, see FIG. 3). The movement of the base may be a rocking motion, a compression motion, a pivoting motion, a rotating motion, or a combination thereof, as outlined above. For example, the agitator may provide a tilting motion to rock the base or a vertical motion to compress the cell culture container.

The first period of time may be from 1 to 60 minutes.

The base ceases to move and is maintained in a stationary position for a second period of time at step 140a. During this step, the base is held in a horizontal position.

The second period of time may be from 1 minute to 5 hours. In one example, the second period of time may be from 1 to 60 minutes. In another example, the second period of time may be from 0.5 to 5 hours. In another example, the second period of time may be from 1 to 3 hours. In another example, the second period of time is 3 hours.

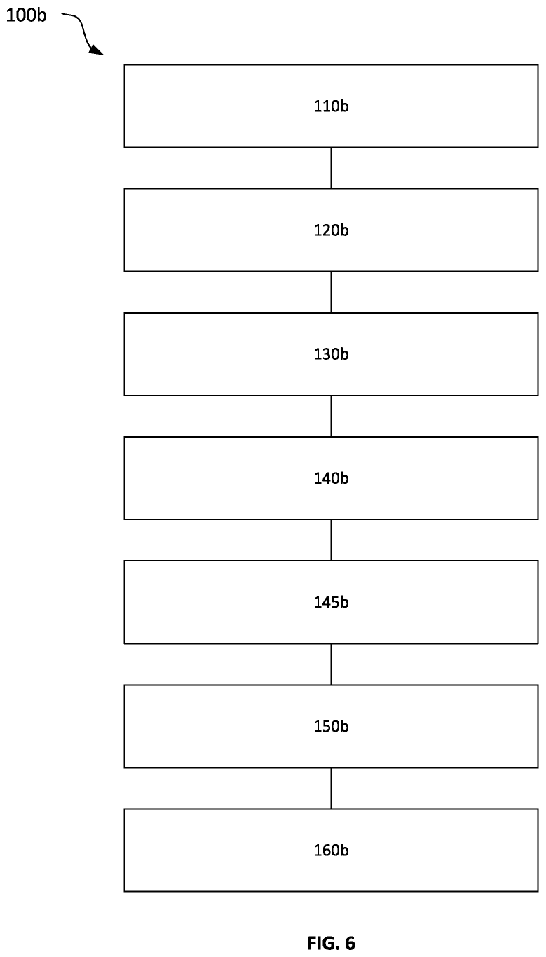

The “intermittent mixing” method is shown in FIG. 6. the base(i.e., base 7, see FIGS. 1 and 2) of the cell culture container is moved with respect to the top (i.e., interface plate 3, see FIGS. 1 and 2) for a first period of time at step 130b. The base may be caused to move by the agitator (i.e., agitator 18, see FIG. 3). The movement of the base may be a rocking motion, a compression motion, a pivoting motion, a rotating motion, or a combination thereof, as outlined above. For example, the agitator may provide a tilting motion to rock the base or a vertical motion to compress the cell culture container.

The first period of time may be from 1 minute to 5 hours. For example, the first period of time may be from 1 to 60 minutes. In another example, the first period of time may be from 0.5 to 5 hours. In another example, the first period of time may be from 1 to 3 hours. In another example, the first period of time may be 1 minute. In another example, the first period of time may be 3 hours.

The agitator ceases movement of the base and maintains the base in a stationary position for a second period of time at step 140b. During this step, the base is held in a horizontal position.

The second period of time may be from 1 minute to 5 hours. For example, the second period of time may be from 1 to 60 minutes. In another example, the second period of time may be from 0.5 to 5 hours. In another example, the second period of time may be from 1 to 3 hours. In another example, the second period of time may be 3 hours.

In one example, the first period of time may be 1 minute and the second period of time may be 3 hours. In another example, the first period of time may be 3 hours and the second period of time may be from 1 to 5 hours.

The above described steps 130b and 140b are repeated for a predetermined period of time at step 145b. This provides intermittent mixing of the cell suspension in the cell culture container (i.e., cell culture container 2, see FIGS. 1 and 2). The predetermined period of time may be from 1 to 5 days. In examples, the predetermined period of time may be from 1 to 3 days. In another example, the predetermined period of time may be 3 days.

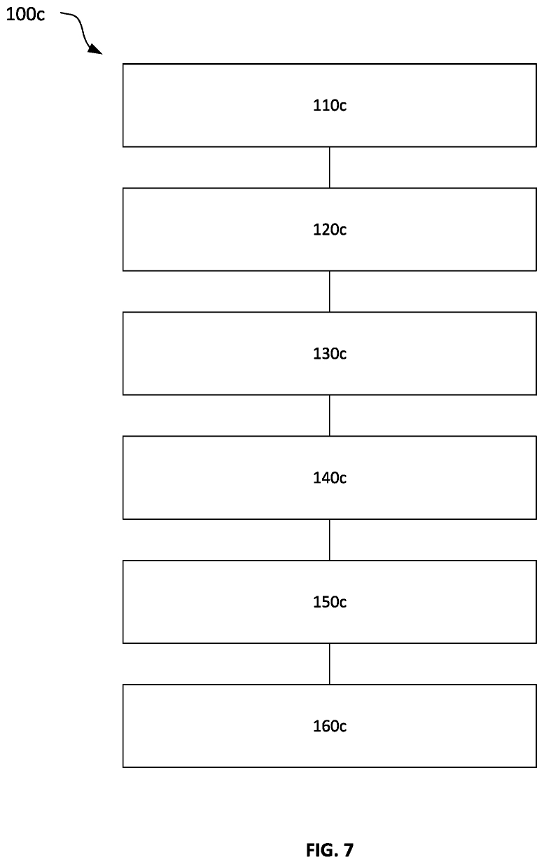

The “continuous mixing” method is shown in FIG. 7. The base (i.e., base 7, see FIGS. 1 and 2) of the cell culture container is moved with respect to the top (i.e., interface plate 3, see FIGS. 1 and 2) for a first period of time at step 130c. The base may be caused to move by the agitator (i.e., agitator 18, see FIG. 3). The movement of the base may be a rocking motion, a compression motion, a pivoting motion, a rotating motion, or a combination thereof, as outlined above. For example, the agitator may provide a tilting motion to rock the base or a vertical motion to compress the cell culture container.

The first period of time may be from 1 to 5 days. In examples, the first period of time may be from 1 to 3 days.

The base ceases to move at step 140c.

Any combination of the quick mixing method, intermittent mixing method, and continuous mixing method may be used during transduction of the cells. For example, the cell solution may be mixed intermittently for a first predetermined period of time by a rocking motion, mixed continuously for a second predetermined period of time by a rocking motion, and mixed continuously for a third predetermined period of time by a compression motion. Additional media can be added into the internal volume between, or during, each of these mixing steps to account for the increasing population of cells.

Following transduction, a sample of the cell solution may be taken to measure transduction efficacy. The sample may be taken 2 to 4 days after adding the T cells to the to the internal volume of the cell culture container 2 (day 0). For example, the sample may be taken 3 days after adding the T cells to the internal volume of the cell culture container (i.e., the sample is taken on day 3). Transduction efficiency of is measured using green fluorescent protein (GFP) or CD19 markers. Specifically, transduction efficiency of CAR T cells is measured using CD19 markers.

In each of the methods 100a, 100b, 100c of FIG. 5 to FIG. 7, cell processing medium is added to the internal volume of the cell culture container (i.e., cell culture container 2, see FIGS. 1 and 2) at step 150a, 150b, 150c. The volume of cell processing medium added may be equal to the volume of cell processing medium in the internal volume of the cell culture container so as to double the volume of cell processing medium in the cell culture container. Additional material may also be added into the internal volume of the cell culture container, such as growth factors, cytokines, magnetic beads, nutrients, or the like.

The additional cell processing medium may be added to the cell culture container, 2 to 4 days after adding the T cells to the to the internal volume of the cell culture container 2 (day 0). For example, the additional cell processing medium may be added 3 days after adding the T cells to the internal volume of the cell culture container (i.e., additional cell processing medium is added on day 3).

In each of the methods 100a, 100b, 100c, the transduced T cells are then cultured in the internal volume of the cell culture container at step 160a, 160b, 160c. The base (i.e., base 7, see FIGS. 1 and 2) of the cell culture container may be moved with respect to the top (i.e., interface plate 3, see FIGS. 1 and 2) during this step to mix the cell solution. The cell solution may be mixed continuously or intermittently.

The T cells may be cultured for up to 10 days after adding the T cells to the internal volume of the cell culture container 2 (day 0). In examples, the T cells are to be cultured for 5 to 10 days after adding the T cells to the internal volume of the cell culture container 2. In one example, the T cells are to be cultured for about 7 days after adding the T cells to the internal volume of the cell culture container 2 (i.e., the T cells are cultured until day 7).

Cell processing medium may be added to the internal volume of the cell culture container at predetermined intervals during this cell culture step. For example, cell processing medium may be added every 24 hours. In examples, the volume of cell processing medium is doubled at each predetermined interval. In other examples, cell processing medium may be added dynamically in response to increasing cell density.

During this step, the base (i.e., base 7, see FIGS. 1 and 2) of the cell culture container may be moved with respect to the top (i.e., interface plate 3, see FIGS. 1 and 2) to provide continuous mixing. The base may be caused to move by the agitator (i.e., agitator 18, see FIG. 3). The movement of the base may be a rocking motion, a compression motion, a pivoting motion, a rotating motion, or a combination thereof, as outlined above. In one example, the base is continuously moved in a rocking motion a first predetermined period of time, then the base is continuously moved in a compression motion for. In examples, each of the first predetermined period of time and the second predetermined period of time is 1 to 4 days, preferably about 2 days.

After a predetermined period of time, the T cells are harvested from the cell culture container (i.e., cell culture container 2, see FIGS. 1 and 2). In examples, the predetermined period of time is 5 to 10 days after adding the T cells to the internal volume of the cell culture container (day 0). In one example, the T cells are harvested about 7 days after adding the T cells to the internal volume of the cell culture container (i.e., the T cells are harvested on day 7). Alternatively, the T cells may be harvested once they are at a target density or target cell count. The harvested T cells may be prepared for infusion into a patient or may be frozen for storage or transport.

### Example 1

### Transduction and Culturing of CD3+ T Cells With CD4+ Markers in a Compressible Container

A test was conducted to evaluate the viability, growth and phenotype of primary CD 3+ T with CD 4+ markers cells activated, transduced, and expanded for 7 days in the compressible container (i.e., cell culture container 2, see FIG. 1) with dynamic mixing. Known bioreactor systems, including a rigid gas-permeable container (G-REX® 100M™, Wilson Wolf), flexible gas-permeable bags (Xuri W25™, Cytiva & VueLife® “C” Series Bags, Saint-Gobain), and a rigid container with a stirrer (Prodigy®, Miltenyi Biotec) were used as controls. For each of the controls, transduction occurred while each bioreactor system remained in a static state. The rigid gas-permeable container remained static throughout the test. The gas-permeable bags remained static during transduction, and then were rocked continuously following the transduction period (from day 3, i.e., during subsequent culture). The contents of the rigid container with the stirrer remained static (i.e., not stirred) during transduction, and then were stirred continuously following the transduction period (from day 3, i.e., during subsequent culture).

CD3+ T cells with CD4+ markers were negatively selected from a healthy donor whole blood sample, and seeded in the cell culture container (i.e., cell culture container 2, see FIG. 1) at a density of 1×10⁶cells. mL⁻¹. The CD3^{+ T} cells were seeded on day 0 in a cell processing medium having a volume of 50 mL. The cell processing medium comprised of X-VIVO™15 (Lonza), 5% Normal human AB serum (Sigma) and rhIL-2 (100 units. mL⁻¹) (R&D Systems). The total number of cells seeded was 50×10⁶ cells. Activation agent, CTS™ Dynabeads™ (ThermoFisher), was added in a 3:1 bead-cell ratio on day 0.

Twenty-four hours after seeding the cells into the bioreactor 1, GFP Lentiviral vector (multiplicity of infection (MOI) of 1) (Takarabio) was added to the cell solution (i.e., on day 1).

The cells were cultured for 7 days and the cell solution was sampled at specified intervals prior to the addition of fresh cell processing medium. The volume of additional cell processing medium (the constituents of which are described above) added to the cell solution was generally equal to the volume of cell processing medium in the cell solution, so as to double the volume each time the cell processing medium was added to the cell culture container. Further details of cell processing medium addition are noted below.

Day 3: 150 mL of cell processing medium (the constituents of which are described above) was added post-sampling.

Day 5: 200 mL of cell processing medium (the constituents of which are described above) added post-sampling.

Day 6: 400 mL of cell processing medium (the constituents of which are described above) added post-sampling.

From day 0 to day 3, the base 7 of the bioreactor 1 was actuated to provide an intermitted rocking motion. The base was rocked five times at a rate of five rocks per minute (rpm) every 3 hours, and the base was maintained in a static position between each set of rocks. The rocking motion was a trapezoidal rocking motion.

From day 3 to day 5, the base (i.e., base 7, see FIGS. 1 and 2) of the cell culture container was actuated to provide a continuous rocking motion at a rate of 5 rpm.

From day 5 to day 7, the base of the cell culture container was actuated to provide a continuous linear compression motion (expansion and contraction of the cell culture container) at a rate of 60 cycles per minute.

On day 7, the test was terminated.

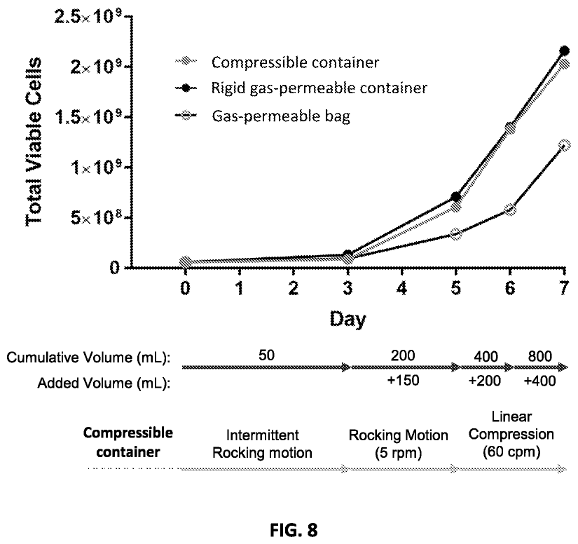

FIG. 8 shows the total number of viable cells over the seven day period of testing. The total number of viable cells is comparable to the control of the static gas-permeable bioreactor container, and there is a greater number of viable cells in comparison with the control of the gas-permeable bags that were static from days 1 to 3 and continuously rocked from days 4 to 7.

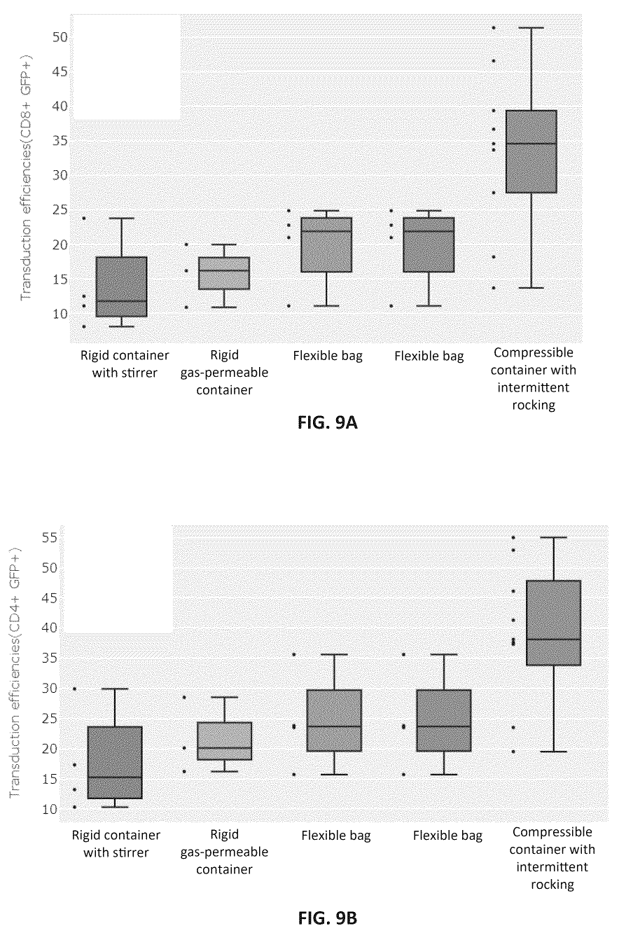

FIG. 9B shows a comparison of the transduction efficiencies of CD4+ T cells using various devices. In this example, GFP was used as a marker for transduction. As shown in FIG. 9B, 13 to 52% of CD4+ T cells were effectively transduced according to the present method.

### Example 2

### Transduction and Culturing of CD3+ T Cells With CD8+ Markers in a Compressible Container

The same methodology and containers were utilized for the transduction and culture of CD3+ T cells with CD8+ markers. The methodology and containers used were identical, except in that a population of CD3+ T cells with CD8+ markers were utilized, and hence shall not be described further.

As shown in FIGS. 9A, 19% to 55% of CD8+ T cells were effectively transduced according to the present method.

Therefore, as shown in the results of FIGS. 9A and 9B, a transduction performed in the bioreactor as described herein and in accordance with the methodology above had a statistically significant greater transduction efficiency when compared with the control systems.

### Example 3

### Transduction and Culturing of CD3+ T Cells With CD4+ Markers

A test was conducted to evaluate the viability, growth and transduction efficiency of primary CD3+ T cells with CD4+ markers and CD3+ T cells with CD8+ markers. The compressible container having a baffle as described herein (see FIG. 2) was compared in three mixing modes (static, mixing mode 1, mixing mode 2, each as defined below) to a rigid gas-permeable container acting as a control. For the control, transduction occurred while the container system remained in a static state (i.e., not rocked or otherwise moved).

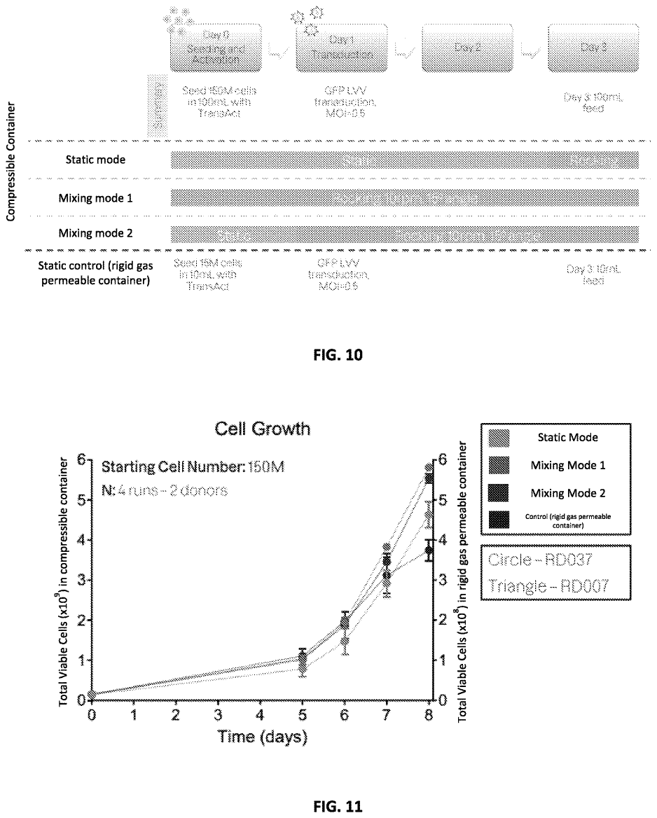

FIG. 10 provides an overview of the respective mixing modes for days 0 to 3 for the compressible container and for the control, which is explained in further detail below.

### Compressible Container

CD3+ T cells with CD4+ markers and CD3+ T cells with CD8+ markers were negatively selected from a healthy donor whole blood sample, and seeded in the cell culture container (i.e., cell culture container 2, see FIG. 2) at a density of 1.5×10⁶cells. mL⁻¹ . The cells were seeded on day 0 in a cell processing medium having a volume of 100 mL. The cell processing medium comprised of TexMACS™ (Miltenyi Biotec), 5% Normal human AB serum (Sigma-Aldrich), IL-7 (12.5 ng·mL⁻¹) (Miltenyi Biotec), and IL-15 (12.5 ng·mL⁻¹) (Miltenyi Biotec). The total number of cells seeded was 150×10⁶ cells. Activation agent, TransAct™ (Miltenyi Biotec), was added in a 1:100 TransAct™-media ratio on day 0.

Twenty-four hours after seeding the cells into the compressible container, GFP Lentiviral vector (multiplicity of infection (MOI) of 0.5) (Flash Therapeutics) was added to the cell solution (i.e., on day 1).

The cells were cultured for 8 days and the cell solution was sampled at specified intervals. The addition of fresh cell processing medium was provided. The volume of additional cell processing medium (the constituents of which are described above) added to the cell solution was generally equal to the volume of cell processing medium in the cell solution, so as to double the volume each time the cell processing medium was added to the cell culture container. Further details of cell processing medium additions and the process are noted below.
Day 0: cell seeding and activation.
Day 1: transduction reagent added (green fluorescent protein (GFP) Lentiviral vector).
Day 3: 100 mL of cell processing medium (the constituents of which are described above) was added.
Day 4: 200 mL of cell processing medium (the constituents of which are described above) was added.
Day 5: 400 mL of cell processing medium (the constituents of which are described above) was added. Prior to cell processing medium addition, a sample was also taken on day 5.
Day 6: 200 mL of cell processing medium (the constituents of which are described above) was added. Prior to cell processing medium addition, a sample was also taken on day 6.
Day 7: A sample was taken. No further cell processing medium was added.
Day 8: A final sample was taken representative of the end of the process.

As noted in FIG. 10, in a static mode of the compressible container, the base 7 of the cell culture container 2 (see FIG. 2) was provided statically in a horizontal position for the duration of the transduction process (i.e., up to day 3). That is, the base 7 of the cell culture container 2 (see FIG. 2) was not moved during this time.

As noted in FIG. 10, in a first mixing mode (“mixing mode 1”) of the compressible container, the base 7 of the cell culture container 2 (see FIG. 2) was actuated to provide a continuous rocking motion on days 0 to 3 (inclusive). The base was rocked continuously at a rate of ten (10) rocks per minute. The rocking motion was a sinusoidal (i.e., smooth or constant velocity) rocking motion. The base 7 was provided at a 15 degree angle to the horizontal during rocking.

As illustrated in FIG. 10, in a second mixing mode (“mixing mode 2”) of the compressible container, the base 7 of the cell culture container 2 (see FIG. 2) was actuated to provide a continuous rocking motion on days 1 to 3 (inclusive), i.e., following the addition of the transduction agent but not prior to its addition. The base was rocked continuously at a rate of ten (10) rocks per minute. The rocking motion was a sinusoidal (i.e., smooth or constant velocity) rocking motion. The base 7 was provided at a 15 degree angle to the horizontal during rocking.

In each of the static modes, mixing mode 1 and mixing mode 2, day 4 incorporated a further continuous rocking motion for twenty-four (24) hours. The base was rocked at a rate of thirty (30) rocks per minute. The rocking motion was a sinusoidal (i.e., smooth or constant velocity) rocking motion. The base 7 was provided at a 15 degree angle to the horizontal during rocking.

In each of the static modes, mixing mode 1 and mixing mode 2 for the compressible container, days 5 to 8 (not shown in FIG. 10) incorporated a further compression mixing regime as outlined below.

On days 5 to 8, inclusive, the base 7 of the cell culture container 2 (see FIG. 2) was actuated to provide a continuous linear compression motion (expansion and contraction of the cell culture container) at a rate of 22 cycles per minute, each expansion (and subsequent contraction) having a stroke length of 20 mm.

On day 8, the test was terminated following a final sampling of the cells from the cell culture container.

### Rigid Gas-Permeable Control Device

For the purposes of a control device, a cell culture container was utilized having a rigid and gas-impermeable cylindrical wall upstanding from a gas permeable base. The gas permeable base is support by a support, such as feet, that enables passive diffusion of gas into the volume of the cell culture container via the gas permeable base. The cell culture container was also provided with an access port and a cap coupled to the access port that was removeable to enable input and removal of materials to the container. Such a container includes a gas permeable base comprised of silicone with a surface area of 10 cm². The container also has a volumetric capacity of 100 mL.

CD3+ T cells with CD4+ markers and CD3+ T cells with CD8+ markers were negatively selected from a healthy donor whole blood sample, and seeded in a rigid gas-permeable container (i.e., a container having rigid walls and a gas permeable base as mentioned above) at a density of 1.5×10⁶cells·mL⁻¹. The cells were seeded on day 0 in a cell processing medium having a volume of 10 mL. The cell processing medium comprised of TexMACS™ (Miltenyi Biotec), 5% Normal human AB serum (Sigma-Aldrich), IL-7 (12.5 ng·mL⁻¹) (Miltenyi Biotec), and IL-15 (12.5 ng·mL⁻¹) (Miltenyi Biotec). The total number of cells seeded was 15×10⁶ cells. Activation agent, TransAct™ (Miltenyi Biotec) was added in a 1:100 TransAct™-media ratio on day 0.

Twenty-four hours after seeding the cells in the rigid gas-permeable container, GFP Lentiviral vector (multiplicity of infection (MOI) of 0.5) (Flash Therapeutics) was added to the cell solution (i.e., on day 1).

The cells were cultured for 8 days and the cell solution was sampled at specified intervals as discussed below.

Day 3: 10 mL of cell processing medium (the constituents of which are described above) was added.

Day 4: 20 mL of cell processing medium (the constituents of which are described above) was added.

Day 5: 40 mL of cell processing medium (the constituents of which are described above) was added and a sample was taken.

Day 6: 20 mL of cell processing medium (the constituents of which are described above) was added and a sample was taken.

Day 7: sample was taken and frozen.

Day 8: the test was terminated following a final sampling of the cells from the device.

The rigid gas permeable container remained static (i.e., was not moved) throughout the duration of the experiment (i.e., across each of days 0 to 8) as shown in FIG. 10.

### Comparison of Compressible Container and Rigid Gas-Permeable container

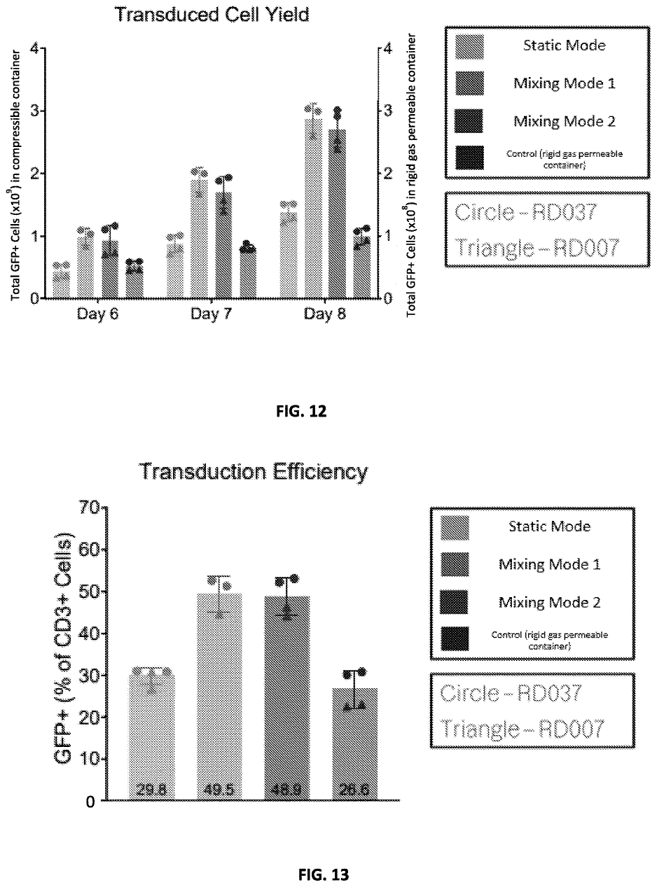

Each experiment was run four times (N=4 for each of the static mode, mixing mode 1, mixing mode 2 and control) for two donors (indicated by RD037 and RD007 in FIG. 12 and FIG. 13 discussed below) and the results compared as outlined in FIG. 11, FIG. 12 and FIG. 13, and as further discussed below.

FIG. 11 shows the total number of viable cells over the period of testing comparing the static mode, mixing mode 1 and mixing mode 2 in the compressible container to that of the rigid gas-permeable container acting as a control. The total viable cells in the compressible container are expressed in 10⁹ cells whereas the total viable cells in the rigid gas permeable container are expressed in 10⁸ as the starting volumes differed by an order of magnitude between such containers (1000 mL in the compressible container; 100 mL in the rigid gas permeable container) due to their volumetric capacities. However, the same seeding densities (1.5×10⁶ cells. mL⁻¹) were provided in each container as outlined above.

As shown in FIG. 11, the total number of viable cells in the compressible container exceeds the control of the rigid gas-permeable bioreactor container in each of the mixing conditions (static mode, mixing mode 1, mixing mode 2). As also shown, the total number of viable cells is enhanced through the provision of mixing conditions in the compressible container (mixing mode 1, mixing mode 2) compared to a static growth conditions (static mode).

FIG. 12 shows a comparison of the day 6 to 8 transduced cell yields. In particular, a statistically significant greater number of GFP+ cells (i.e., cells taking up the GFP lentiviral vector) were obtained in the compressible bioreactor on days 7 and 8 when utilizing mixing mode 1 and mixing mode 2 in comparison to the rigid gas-permeable container control (P-value<0.0001). It is also noted that mixing mode 1 and mixing mode 2 also outperformed the control at day 6. Additionally, as shown in FIG. 11, the static mode of the compressible container also outperformed the control on days 7 and 8.

FIG. 12 also illustrates that the end-to-end process time may be reduced by as much as two days. In particular, utilizing mixing mode 1 or mixing mode 2 in a compressible container disclosed herein achieves a transduced cell yield on day 6 comparative to that achieved on day 8 in the rigid gas-permeable bioreactor.

FIG. 13 shows a comparison of the transduction efficiencies between the compressible container and the rigid gas-permeable container control, and also compares the various mixing profiles in the compressible container. As can be seen, the compressible container is comparable to the rigid gas-permeable bioreactor when utilized in a static mode. As also can be seen, the compressible container shows a statistically significant improvement in transduction efficiency when utilized in mixing mode 1 or mixing mode 2 in comparison to the rigid gas-permeable container control and the static mode of transduction in the compressible container. The transduction efficiency for each experiment was assessed on each of days 6 to 8 (inclusive), and was assessed using a flow cytometer (specifically a FACSLyric™ device by BD Biosciences).

Therefore, it has been shown that the provision and use of a compressible container can enhance transduction efficiencies, reduce the end-to-end cell processing time, and enhance total viable cell numbers.

Throughout the description and claims of this disclosure, the words “comprise” and “contain” and variations of them mean “including but not limited to,” and they are not intended to (and do not) exclude other components, integers or steps. Throughout the description and claims of this disclosure, the singular encompasses the plural unless the context otherwise requires. In particular, where the indefinite article is used, the disclosure is to be understood as contemplating plurality as well as singularity, unless the context requires otherwise.

Features, integers, characteristics, or groups described in conjunction with a particular aspect, embodiment or example of the disclosure are to be understood to be applicable to any other aspect, embodiment or example described herein unless incompatible therewith. All of the features disclosed in this disclosure (including any accompanying claims, abstract and drawings), and/or all of the steps of any method or process so disclosed, may be combined in any combination, except combinations where at least some of such features and/or steps are mutually exclusive. The disclosure is not restricted to the details of any foregoing embodiments. The disclosure extends to any novel one, or any novel combination, of the features disclosed in this disclosure (including any accompanying claims, abstract and drawings), or to any novel one, or any novel combination, of the steps of any method or process so disclosed.

1. A method of transducing T cells, comprising:
providing a compressible container including a base, a top arranged substantially in parallel to the base, and at least one flexible wall element extending between the top and the base and defining an internal volume of the compressible container;
adding a population of T cells in a cell processing medium into the internal volume;
adding a transduction agent into the internal volume; and
moving the base with respect to the top, thereby causing turbulence of contents in the internal volume, for a first period of time.

2. The method of claim 1, further comprising moving the base with respect to the top, thereby causing turbulence of contents in the internal volume, for a second period of time.

3. The method of claim 2, wherein moving the base with respect to the top for the first period of time and/or moving the base with respect to the top for the second period of time comprises rotating the base about an axis of rotation extending within a horizontal plane defined by the base.

4. The method of claim 1, wherein the base is moved with respect to the top for the first period of time at a first rate, and wherein the base is moved with respect to the top for the second period of time at a second rate, the second rate being greater than the first rate.

5. The method of claim 1, further comprising moving the base with respect to the top, thereby causing turbulence of contents in the internal volume, for a third period of time.

6. The method of claim 5, wherein moving the base with respect to the top for the third period of time comprises translating the base toward the top along a central longitudinal axis of the compressible container, thereby compressing the compressible container.

7. The method of claim 1, further comprising maintaining the base stationary with respect to the top for a second period of time.

8. The method of claim 7, wherein the second period of time is 1 to 5 hours, preferably about 3 hours.

9. The method of claim 7, further comprising moving the base with respect to the top, thereby causing turbulence of contents in the internal volume, for a third period of time.

10. The method of claim 9, wherein third period of time is 1 to 60 minutes.

11. The method of claim 7, wherein the, either, or both, of the steps of moving the base with respect to the top comprises rotating the base about an axis of rotation extending within a horizontal plane defined by the base.

12. The method of claim 3, wherein rotating the base comprises:
rotating the base in a first direction about the axis of rotation into a first position in which the base forms a first angle with respect to the horizontal plane; and
rotating the base in a second direction, opposite to the first direction, about the axis of rotation into a second position in which the base forms a second angle with respect to the horizontal plane.

13. The method of claim 12, wherein the base is rotated between the first position and the second position at a constant velocity.

14. The method of claim 12, wherein the base is rotated between the first position and the second position at a varying velocity.

15. The method of claim 12, wherein the base is maintained in the first position and/or the second position for a predetermined period of time.

16. The method of claim 12, wherein the base is rotated between the first position and the second position at a rate of 1 to 30 rotations per minute, preferably 5 to 25 rotations per minute, more preferably 10 to 20 rotations per minute.

17. The method of claim 12, wherein the first angle and/or the second angle is 1 to 45 degrees, preferably 5 to 25 degrees.

18. The method of claim 1, wherein the, either, or both, of the steps of moving the base with respect to the top comprises translating the base toward the top along a central longitudinal axis of the compressible container, thereby compressing the compressible container.

19. The method of claim 6, wherein the compressible container is compressed at a rate of 1 to 60 compressions per minute, preferably 20 to 40 compressions per minute.

20. The method of claim 1, wherein the, either, or both, of the steps of moving the base with respect to the top comprises pivoting the base about a centrally disposed origin of the base.

21. The method of claim 20, wherein the base is pivoted at a rate of 1 to 30 revolutions per minute, preferably 5 to 25 revolutions per minute, more preferably 10 to 20 revolutions per minute.

22. The method of claim 1, wherein the transduction agent is added into the internal volume during the step of moving the base with respect to the top for the first period of time.

23. The method of claim 1, further comprising the step of maintaining the base stationary with respect to the top following the addition of the transduction agent for a predetermined period of time.

24. The method of claim 9, wherein the transduction agent is added into the internal volume during the step of moving the base with respect to the top for the third period of time.

25. The method of claim 1, wherein the transduction agent is a viral vector, such as a lentiviral vector.

26. The method of claim 1, wherein the flexible wall element comprises a wall having one or more folds, such as Z-folds.

27. The method of claim 1, wherein the compressible container is substantially gas-impermeable.

28. The method of claim 1, wherein the compressible container is at least partially gas-permeable.

29. The method of claim 28, wherein the base and/or the flexible wall element comprises a gas-permeable material, such as silicone or fluorinated ethylene propylene.

30. The method of claim 1, wherein the T cells are selected from CD3+ T cells having CD4+ markers and/or CD3+ T cells having CD8+ markers.

31. A method of transducing T cells, comprising the ordered steps of:
i) providing a compressible container including a base, a top arranged substantially in parallel to the base, and at least one flexible wall element extending between the top and the base and defining an internal volume of the compressible container;
ii) adding a population of T cells in a cell processing medium into the internal volume;
iii) continuously moving the base with respect to the top, thereby causing turbulence of contents in the internal volume, at a first rate for a first period of time;
iv) optionally adding further cell processing medium into the internal volume;
v) continuously moving the base with respect to the top, thereby causing turbulence of contents in the internal volume, at a second rate for a second period of time, the second rate being greater than the first rate;
vi) optionally adding further cell processing medium into the internal volume; and
vii) continuously moving the base with respect to the top, thereby causing turbulence of contents in the internal volume, for a third period of time,
wherein the method further comprises the step of adding a transduction agent into the internal volume during step ii) or step iii).

32. The method of claim 31, wherein:
step iii) comprises continuously rotating the base about an axis of rotation extending within a horizontal plane defined by the base at the first rate;
step v) comprises continuously rotating the base about an axis of rotation extending within a horizontal plane defined by the base at the second rate; and
step vi) comprises continuously translating the base toward the top along a central longitudinal axis of the compressible container, thereby compressing the compressible container.

33. A method of transducing T cells, comprising the ordered steps of:
i) providing a compressible container including a base, a top arranged substantially in parallel to the base, and at least one flexible wall element extending between the top and the base and defining an internal volume of the compressible container;
ii) adding a population of T cells in a cell processing medium into the internal volume;
iii) optionally maintaining the base stationary with respect to the top following the addition of the a transduction agent for a predetermined period of time;
iv) moving the base with respect to the top, thereby causing turbulence of contents in the internal volume, for a first period of time;
v) maintaining the base stationary with respect to the top for a second period of time;
vi) repeating steps iv) and v) for a predetermined period of time; and
vii) adding a transduction agent into the internal volume during any of steps ii) to vi).

34. A method of transducing and culturing T cells, comprising the ordered steps of:
i) providing a compressible container including a base, a top arranged substantially in parallel to the base, and at least one flexible wall element extending between the top and the base and defining an internal volume of the compressible container;
ii) adding a population of T cells in a cell processing medium into the internal volume;
iii) optionally maintaining the base stationary with respect to the top following the addition of the transduction agent for a predetermined period of time;
iv) intermittently moving the base with respect to the top, thereby intermittently causing turbulence of contents in the internal volume, for a first period of time;
v) adding a transduction agent into the internal volume during the step of adding a population of T cells in the cell processing medium into the internal view, or during the step of intermittently moving the base with respect to the top to enable transduction of the population of T cells;
vi) adding an additional volume of cell processing medium into the internal volume; and
vii) culturing the population of T cells.

35. A method of transducing and culturing T cells, comprising the ordered steps of:
i) providing a compressible container including a base, a top arranged substantially in parallel to the base, and at least one flexible wall element extending between the top and the base and defining an internal volume of the compressible container;
ii) adding a population of T cells in a cell processing medium into the internal volume;
iii) optionally maintaining the base stationary with respect to the top following the addition of the transduction agent for a predetermined period of time;
iv) rotating the base about an axis of rotation extending within a horizontal plane defined by the base, thereby causing turbulence of contents in the internal volume, for a first period of time;
v) maintaining the base stationary with respect to the top for a second period of time;
vi) repeating steps iv) and v) for a predetermined period of time;
vii) optionally adding additional cell processing medium into the internal volume;
viii) translating the base with respect to the top along a central longitudinal axis of the compressible container, thereby compressing the compressible container, for a third period of time; and
ix) adding a transduction agent into the internal volume during any of steps ii) to viii).
